(19) United States
(12) Patent Application Publication
(10) Pub. No.: US 2018/0274007 A1
(43) Pub. Date: Sep. 27, 2018

(54) METHODS OF GENOME SEQENCING AND EPIGENETIC ANALYSIS

(71) Applicant: CARNEGIE INSTITUTION OF WASHINGTON, Washington, DC (US)

(72) Inventors: Yixian ZHENG, Washington, DC (US); Junling JIA, Washington, DC (US); Xiaobin ZHENG, Washington, DC (US)

(21) Appl. No.: 15/758,032

(22) PCT Filed: Sep. 14, 2016
(86) PCT No.: PCT/US16/51599

§ 371 Date: Mar. 7, 2018

Related U.S. Application Data
(63) Continuation of application No. 14/853,250, filed on Sep. 14, 2015, now abandoned.

(51) Int. Cl. C12Q 1/6806

(52) U.S. Cl. CPC C12Q 1/6806; C12Q 2563/149; C12Q 2535/113

(57) ABSTRACT

Novel methods of ChIP-seq are disclosed herein. These methods of ChIP-seq employ carrier DNA to prevent loss of DNA samples. The greater DNA yields achieved by this invention permit ChIP-seq of a small number of cells, permitting epigenetic analysis of primary cells of limited quantity.

The present application claims benefit of U.S. patent application Ser. No. 14/853,250, filed Sep. 14, 2015, the entire contents of which is incorporated herein by reference. U.S. patent application Ser. No. 14/853,250, is a continuation-in-part of International Application No. PCT/US2014/026939, filed Mar. 14, 2014. International Application No. PCT/US2014/026939 claims benefit of U.S. Provisional Patent Application No. 61/790,320, filed Mar. 15, 2013.

### FIELD OF THE INVENTION

This invention relates to novel methods of genome sequencing and epigenetic analysis.

### BACKGROUND

The epigenetic state of chromatin regulates the access of transcription factors and the replication machinery to DNA. In eukaryotes, factors that regulate the epigenetic state of a cell are, for example, methylation of DNA and covalent modifications to histones. The development of next-generation sequencing, has made it possible to obtain profiles of epigenetic modifications across a genome using chromatin immunoprecipitation (ChIP-seq). ChIP-seq allows high resolution detection of proteins that bind to specific regions of the genome and it can be used to pinpoint epigenetic modifications that lead to phenotypic changes within a cell.

Epigenetic modifications refer to reversible, covalent modifications to specific DNA sequences and their associated histones. These reversible, covalent modifications influence how the underlying DNA is utilized and can therefore also control traits (Jenuwein and Allis (2001) Science, 293, 1074-1080; Klose and Bird (2006) Trends In Biochemical Sciences, 31, 89-97).

Epigenetic modifications to the mammalian genome include methylation, acetylation, ribosylation, phosphorylation, sumoylation, citrullination, and ubiquitylation. These modifications can occur at more than 30 amino acid residues of the four core histones within the nucleosome. For example, the most common epigenetic modifications to DNA in mammals are methylation and hydroxymethylation of DNA, both of which may be made on the fifth carbon of the cytosine pyrimidine ring.

Epigenetic modifications to the genome can influence development and health as profoundly as mutagenesis of the genome. Specifically, the epigenetic modifications described above do not alter the primary DNA sequence. Rather, the epigenetic modifications have a potent influence on how underlying DNA is expressed. As a result, epigenetic modifications can alter phenotypes as powerfully as mutations in a DNA sequence.

For example, mutations to the p16 tumor suppressor gene (i.e., mutations in the nucleotide sequence) silences the gene. Similarly, methylation of DNA at the promoter of the p16 tumor suppressor gene (i.e., no mutations to the nucleotide sequence) silences the gene. Both events (i.e., the mutations to the nucleotide sequence and the methylation of the correct sequence) contribute to the development and progression of colorectal cancer. However, unlike mutations which are permanent, epigenetic silencing of p16 can be reversed pharmacologically. Accordingly, the ability to detect epigenetic modifications provides an avenue for medical intervention and directed treatment plans.

Specific epigenetic modifications that occur genome wide also regulate cellular differentiation during development (Mikkelsen et al. (2007) Nature, 448, 553-U552). For example, epigenetic modifications in mature tissues contribute to initiation and progression of cancer and other diseases (Feinberg, A. P. (2007) Nature, 447, 433-440). Additionally, studies have shown that epigenetic modifications are influenced by environmental variables including diet (Waterland and Jirtle (2003) Molecular And Cellular Biology, 23, 5293-5300), environmental toxins (Anway et al. (2005) Science, 308, 1466-1469) and maternal behaviors (Weaver et al. (2004) Nature Neuroscience, 7, 847-854). Given the fundamental role that epigenetic modifications play in normal development, environmental responses, disease development, and disease progression, there is need to develop methods of sequencing genomic DNA to detect epigenetic modifications. Specifically, there is a need to develop methods of sequencing genomic DNA to detect epigenetic modifications from a small number of cells that can be obtained by a simple biopsy or tissue sample.

Furthermore, even though epigenetic modifications do not consist of changes to the DNA sequence, they can be passed from mother to daughter cells during mitosis and they can persist through meiosis to be transmitted from one generation to the next. Accordingly, even though epigenetic modifications can change and revert to their original state far more readily than changes to a DNA sequence, they remain fundamental to development and disease.

Epigenetic modifications have been most notably studied as they relate to cancer development and cancer progression. For example, early observations linked perturbations in DNA methylation to the development of human colorectal cancer and subsequent studies showed that experimental manipulation of DNA methylation state, pharmacologically or genetically, have the power to control tumor development. Accordingly, a growing area of research shows that therapies directed at modifying epigenetic states can control cancer and disease progression. Likewise, epigenetic modifications can be mapped to disease states and can be used as biomarkers to detect or prevent disease development and progression.

Other examples of epigenetic modifications are those that develop in response to an organism's environment (e.g., where a human lives and what the human is exposed to in the surrounding environment can influence epigenetic modifications). Examples of environmental factors that influence epigenetic include maternal behavior during nursing, exposure to endocrine disruptors, and the nutrient composition of diets. Furthermore, as described above, epigenetic modifications and resulting phenotypes, can be transmitted from parent to offspring, even if only the parents and not the offspring are exposed to the environmental factors. This raises the possibility that some complex traits that run in families, like obesity, cancer or behavioral patterns, are transmitted through epigenetic modifications and result from the exposure environmental factors experienced during prior generations.

Existing approaches for analyzing epigenetic modifications of chromatin, such as chromatin immunoprecipitation (ChIP), are labor-intensive and require serial processes that impose significant limitations on analysis throughput and sample quantity.

ChIP involves immunoprecipitation using an antibody specific to epigenetic modifications of interest to isolate modified chromatin, which is subsequently analyzed using massive parallel DNA sequencing (ChIP-seq), microarray hybridization or gene-specific PCR. ChIP can be used to characterize the genome placement of a chromatin associated protein and is the predominant analytical tool currently practiced in epigenomic and chromatin research. However, it suffers from major limitations. First, the analysis generally requires at least 10⁷ cells. In other words, current ChIP methods require far too many cells than are available to study epigenetic modifications and changes when cell numbers are limited. For example, it is not possible to perform ChIP-seq on embryos, primary cells that are not propagated in in vitro culture, microdissected cells, and small cell samples acquired directly from biopsy of a living animal such as a human. Accordingly, current methods for epigenomic testing involve bulk cell analysis (i.e., on average of at least 10⁶ cells).

Genome-wide sequencing of RNA and DNA in a single mammalian cell holds great promise to reveal global transcriptional program and DNA variations with un-precedent accuracy. An important missing link, however, is the information of the epigenetic and transcription factor-binding landscapes of the genome in a small number of cells (e.g., less than 10⁶ cells, for example between 1 and 20,000 cells) dissected from tissues. Multiple steps required for obtaining DNA for deep sequencing has limited the application of chromatin-immunoprecipitation (ChIP) because deep sequencing typically requires large amounts of DNA which cannot be harvested using traditional ChIP methods (i.e., because ChIP requires a number of purification steps, large amounts of DNA are typically lost).

Described herein is a new method based on enhanced recovery of DNA. Specifically, the methods provided herein describe enhancing DNA recovery during ChIP (i.e., preventing DNA loss from purification and processing steps) by the addition of protection agents and favored DNA amplification (RepFamp). These methods allow robust and reliable mapping of epigenetic landscape in a very small number of cells and results in a new and novel method for global transcriptome analysis without cell counting to uncover epigenetic changes.

### BRIEF SUMMARY OF THE INVENTION

The invention relates to methods of sequencing genomic DNA from a sample of cells, with the methods comprising fragmenting chromatin in the sample of cells, adding a carrier DNA to the fragmented chromatin of the sample of cells, where the carrier DNA, termed “DNA1,” is 5′ biotinylated DNA, precipitating the mixture of carrier DNA1 and fragmented chromatin, annealing a blocking primer, which prevents amplification of the DNA and is complementary to the DNA1, amplifying the genomic DNA from the sample of cells, and sequencing the amplified DNA. The methods can be performed on a sample of cells between 1 and 20,000 cells.

The invention relates to methods of sequencing genomic DNA from a sample of cells, with the methods comprising fragmenting chromatin in the sample of cells, adding a carrier DNA, termed “DNA2,” to the fragmented chromatin of the sample of cells, where the carrier DNA is 5′ biotinylated with a 5′ overhang and a 3′ spacer 3 modification, precipitating the mixture of carrier DNA2 and fragmented chromatin, amplifying the genomic DNA from the sample of cells, and sequencing the amplified DNA. The methods can be performed on a sample of cells between 1 and 20,000 cells.

The invention relates to methods of sequencing genomic DNA from a sample of cells, with the methods comprising combining the sample of cells with a collection of bulking cells, fragmenting chromatin in the sample cells and the bulking cells, precipitating the fragmented chromatin of the cells, amplifying the genomic DNA from the sample of cells, and sequencing the amplified DNA. The methods can be performed on a sample of cells between 1 and 20,000 cells.

### BRIEF DESCRIPTION OF THE DRAWINGS

FIG. 1 depicts cartoon illustration comparing (1) Recovery via Protection (RePro, or RP-ChIP-seq) and (2) Recovery via Protection and Favored amplification (RePam, or FARP-ChIP-seq). For RP-ChIP-seq and FARP-ChIP-seq, protection oligomers such as DNA are added to sample cell(s) for ChIP DNA isolation, whole genome DNA isolation, or RNA isolation. In the RP-ChIP-seq scheme both carrier DNA and sample DNA will be amplified (unbiased), which requires an increase in sequencing depth. In FARP-ChIP-seq, specific carrier sequences or PCR primers used inhibit the amplification of the carrier DNA, while allowing the amplification of the DNA of interest. This biased amplification reduces the sequencing depth required. After sequencing, software will be used to filter out reads from carrier DNA to generate reads from the DNA of interest.

FIG. 2 depicts a table listing three of the many possible types of carrier DNAs (genomic DNA from S. cerevisia or E. coli, or synthetic DNA oligo) that come from and their potential of use in genomic studies of Drosophila melanogaster, Mus musculus, and Homo sapiens. The numbers of short sequence tags in the carrier DNA that can be mapped to the genomes of interest are listed. The theoretical short sequence tags are 50 bp long covering the carrier DNA with 1 bp step-length and mapped to the target genome using bowtie allowing 3 mismatches. The use of genomic DNAs from other species allows RePro, while the use of synthetic DNA allows both RePro and RePam. RePam offers favored amplification of DNA of interest by blocking the amplification of carrier DNA and reduces sequencing depth needed for mapping.

FIG. 3 depicts two types of carrier DNA. FIG. 3A depicts carrier DNA1. Carrier DNA1 is biotinylated DNA with a known sequence. FIG. 3B depicts carrier DNA 2. Carrier DNA2 contains the same biotinylated DNA as in DNA1 and an extra 5′ overhang and 3′ Spacer3 modification on both ends. This end structure blocks DNA polymerase to fill in the overhang, so adapter DNA for PCR cannot be ligated to these ends and amplification cannot take place.

FIG. 4 depicts graphs of PCR amplification of carrier DNA1 in the presence and absences of an amplification blocker. The carrier DNA1 is biotinylated double stranded DNA as shown in FIG. 3A. The amplification blocker is a DNA oligo carrying the indicated modifications at the 5′ end. The Bioanalyzer plots show the increase in the blocking of carrier DNA1 amplification with increasing concentration of amplification blocker in the standard library construction procedures. Red arrows indicate the peak of amplified carrier DNA1.

FIG. 5 depicts the demonstration of PCR amplification block of carrier DNA2. The carrier DNA2 is biotinylated double stranded DNA with 3′ modifications as shown in FIG. 3B. Such DNA cannot be ligated to PCR primers used in the library construction, consequently, it cannot be amplified as shown by the lack of the specific DNA2 peaks in the Bioanalyzer plots before and after PCR amplification using standard library construction procedures.

FIG. 6 depicts ChIP-Seq from 500 embryonic stem cells (ESCs) by applying the yeast genomic DNA as a carrier using RePro. FIG. 6A depicts a heatmap showing enrichment of H3K4me3 on gene promoters from 107, 2000, or 500 ESCs. Each line represents one gene. The heatmaps are ranked according to the H3K4me3 enrichment in the 10⁷ cell sample. FIG. 6B depicts contour plots showing the correlation of H3K4me3 enrichment on promoters between the 10⁷ cell sample and the 2000 cell or 500 cell sample with different sequencing depth. Each point represents one gene. The correlation coefficients are spearman correlation. FIG. 6C and FIG. 6D depict the genomic view of ChIP-Seq enrichment of H3K4me3 in the 500 cell, 2000 cell or 10⁷ cell samples in zoomed-out (C) and zoomed-in views (D) along chromosome 17. The peak-height corresponds to RPKM (Reads per Kilo-base per million reads) values calculated in 500 bp windows sliding every 100-bp along the chromosome.

FIG. 7 depicts the proper processing of RNA-Seq reads using the triple normalization method. A mixture of DNA and RNA with known ratio and known sequences are spiked into a sample of cell(s). DNA and RNA isolation and sequencing are performed using standard protocols. The DNA-Seq requires the detection of a fraction of the genomic DNA and the spiked-in DNA reads and therefore only need a very low sequencing depth. The RNASeq following the standard procedure will yield both the reads for RNA from the cell and the spiked-in RNA. The triple normalization scheme shown allows accurate determination of cellular RNA reads without prior knowledge of the cell number used.

FIG. 8 depicts the application of triple normalization method for proper quantification of transcriptional inhibition by Myc inhibitors in ESCs. Heatmaps show analyses of RNASeq fold change based on different normalization strategies. TMM Normalization, the commonly used normalization in the edgeR software package based on the hypothesis that the expression of the majority of genes remains unchanged between different samples, which is incorrect if transcription factors such as Myc is inhibited. Double normalization, normalization using reads of spiked-in RNA and total reads from the sample's genomic DNA. The same percentages of DNA prepared from different samples were loaded for DNA-Seq. Although normalizing against cell's genomic DNA circumvents the need for cell number count, this double normalization fails to avoid variations introduced during library preparation and sequencing. Triple normalization, the normalization procedure described in this patent as illustrated in FIG. 6 above. Only the triple normalization method faithfully demonstrates the global transcriptional inhibition caused by the Myc inhibitor (10058-F4) in ESCs without prior knowledge of the cell number in the samples.

FIG. 9 depicts the analyses of dissected mouse lens epithelial cells to illustrate the application of the invention. Cartoons are drawn to show the eye with lens epithelial cells, which supply the lens fibers and regulate the homeostasis of the lens throughout the mammalian life. Eye diseases such as cataract, which are mostly age-associated, can result from aging-associated changes in the lens epithelial cells. Epigenetic information (such as the status of H3K4me3 modification) will not only shed light on which known pathways (such as electrolyte homeostasis, apopotosis, and cell proliferation) are sensitive to aging but also uncover new pathways that contribute to eye disease.

FIG. 10 depicts graphs showing that RePro enables the high quality mapping of H3K4me3 from a few lens epithelial cells dissected from a single young or old mouse eye. FIG. 10A depicts heatmaps showing enrichment of H3K4me3 on gene promoters of the lens sample from young (post-natal day 30, P30) and old (P800) mice. Each line represents one gene. The heatmaps are ranked according to the H3K4me3 enrichment in the P30 sample. FIG. 10B depicts contour plots showing the good global correlation of H3K4me3 enrichment on promoters between the lens samples of P30 and P800 mice. Each point represents one gene. The correlation coefficient is spearman correlation.

FIG. 11 depicts the identification of aging-associated epigenetic changes in the aging lens epithelial cells. Although the global epigenetic landscapes are similar, the high quality H3K4me3 ChIP-Seq allowed the mapping of significant H3K4me3 modification changes at specific genes. Genes in the indicated functional groups that exhibit significant loss or increase of H3K4me3 modification are shown.

FIG. 12 depicts an example of a simulation demonstrating the number of cells needed in order to attain optimum results using RePro and RePam-ChIP-seq.

FIG. 13 depicts a comparison between RePro-ChIP-seq, LinDA-ChIP-seq, and Nano-ChIP-seq.

### DEFINITIONS

For convenience, the meaning of certain terms and phrases used in the specification, examples, and appended claims, are provided below.

As used herein, the term “a small number of cells” refers to 1 to 100,000 cells. In certain embodiments the term is used to refer to 1 to 20,000 cells, or 1 to 10,000 cells, or 1 to 5,000 cells.

As used herein, the term “RePro” or “Recovery via Protection” refers to a method wherein both carrier DNA and sample DNA are amplified (unbiased), which requires an increase in sequencing depth.

As used herein, the term “RePam” or “Recovery via Protection and Favored amplification” refers to a method wherein specific carrier DNA (referred to as DNA2 herein) is used to inhibit the amplification of the carrier DNA, while allowing the amplification of the DNA of interest. This biased amplification reduces the sequencing depth required.

As used herein, the term “DNA1” refers to 5′ biotinylated carrier DNA.

As used herein, the term “DNA2” refers to 5′ biotinylated carrier DNA which also contains an extra 5′ overhang and 3′ Spacer3 modification on both ends. This end structure blocks DNA polymerase to fill in the overhang, so adapter DNA for PCR cannot be ligated to these ends and amplification cannot take place.

As used herein, the term “epigenetic” refers herein to the state or condition of DNA with respect to changes in function without a change in the nucleotide sequence. Such changes are referred to in the art as “epigenetic modifications,” and tend to result in expression or silencing of genes. Examples of epigenetic changes or marks, which may be caused by modification of DNA in the sample, or of proteins associated with it, and which may be analysed using the method according to the invention include but are not limited to histone protein modification, non-histone protein modification, and DNA methylation.

As used herein, the term “epigenetic analysis” refers to determining the state, or condition of DNA, and its interaction with specific proteins and their modified isoforms in the analyte sample, and involves analysing or detecting epigenetic marks in the analyte biological sample.

As used herein, the term “chromatin immunoprecipitation” will also be known to the skilled technician, and comprises the following three steps:—(i) isolation of chromatin to be analysed from cells; (ii) immunoprecipitation of the chromatin using an antibody; and (iii) DNA analysis. The analyte biological sample, which is subjected to chromatin immunoprecipitation, may comprise chromatin. Chromatin is the substance of a chromosome and includes a complex of DNA and protein (primarily histone) in eukaryotic cells and is the carrier of the genes in inheritance. Chromatin generally occurs in two states, euchromatin and heterochromatin, with different staining properties, and during cell division it coils and folds to form the metaphase chromosomes. Hence, the analyte biological sample comprises nucleic acid, such as but not limited to DNA, and any associated proteins.

The chromatin under analysis can, but need not, be obtained from at one cell. In one embodiment, therefore, the biological sample comprises at least one cell. The cell may be derived from a tissue sample. In certain examples, the cell is derived from a living organism and is not immortalized or propagated in in vitro culture. In certain embodiments the analyte biological sample comprises mammalian cells. In a specific embodiment the analyte biological sample comprises human or mouse cells.

As used herein, the term “suitable primers” refers to chosen primers that can be used for species-specific PCR, i.e. the primers can be used in a PCR that results in the amplification of a length of nucleic acid only from the analyte biological sample, but not from the carrier DNA. Further information regarding the design of suitable primers is provided in the accompanying examples

As used herein, the term “blocking primers” refers to DNA sequences that are complementary to the ends of DNA1. The blocking primers, by annealing to the DNA1 during RePro, prevent PCR amplification of the DNA1. The individual nucleotides (bases) of the blocking primers may or may not have modifications that can prevent their degradation. For example, at least 1, 2, 3, 4, 5, 6, 7, 8, 9, 10 or more bases may be phosphorothioated. Typically, the modifications to the blocking primers occur on one end of the primer. In one specific embodiment, the modification to the blocking primer occurs on the 5′ end of the primer.

As used herein, the term “epigenetic signature” refers to any manifestation or phenotype of cells of a particular cell type that is believed to derive from or can be attributed to chromatin structure (i.e., determined by epigenetic modifications) of such cells.

As used herein, the term “3′ Spacer 3” refers to a three-carbon spacer that is used to incorporate a short spacer arm into an oligonucleotide. The 3′ Spacer 3 can be incorporated into one or more consecutive additions if a longer spacer is required.

As used herein, the term “cells of interest” refers to the cells that contain the DNA to be sequenced using ChIP-seq methods described herein.

As used herein, the term “bulking cells” refers to the addition of cells (e.g., yeast or E. coli cells) to the cells of interest during a ChIP-seq assay. Specifically, bulking cells are added to the cells of interest prior to the sonication and chromatin fragmentation step in the ChIP assay.

As used herein, the term “an agent that specifically binds the DNA” refers to any biological or chemical moiety that binds a DNA of interest. Specifically, as used herein, the DNA of interest is the DNA that is sequenced using the ChIP-seq methods disclosed herein.

As used herein, the terms “analyte biological sample” and “DNA of interest” refer to the DNA that is subject to investigation. In other words, the terms refer to the DNA that is analyzed for epigenetic modifications, epigenetic signatures, and DNA sequencing.

As used herein, the term “chromatin immunoprecipitation” and “ChIP” generally refer to the process comprising the (1) isolation of chromatin to be analysed from cells; (2) immunoprecipitation of the chromatin using an antibody; and (3) DNA analysis.

As used herein, the term “chromatin” refers to the substance of a chromosome and consists of a complex of DNA and protein (primarily histone) in eukaryotic cells, and is the carrier of the genes in inheritance. Chromatin generally occurs in two states, euchromatin and heterochromatin, with different staining properties, and during cell division it coils and folds to form the metaphase chromosomes.

As used herein, the term “carrier DNA” refers to the DNA which is added to act as a bulking agent.

### DETAILED DESCRIPTION OF THE INVENTION

### Introduction

The ability to perform genome wide mapping of transcription factor binding and epigenetic modification in a pure cell population is critical in both basic and translational research. Yet, because chromatin immunoprecipitation (ChIP) followed by massive parallel sequencing (ChIP-seq) requires multi-step manipulations, massive DNA loss has made it impossible to perform ChIP-seq using a small number of cells. Currently, a reliable ChIP-seq experiment requires approximately 50 ng of DNA recovered from ChIP, which generally requires at least 10⁶ cells. Accordingly, it has not been possible to obtain reliable genome wide transcription factor/chromatin protein binding or epigenetic information for basic research and clinical studies using cells of limited quantity (e.g., cells from an embryo, cells from a biopsy, or cells from an eye lens).

Two recent methods have been developed to overcome the difficulty of genome mapping of epigenetic modifications associated with ChIP. Both methods rely on optimizing ChIP and modifying DNA amplification procedures to produce sufficient amount of DNA for sequencing. The first of these method reports the ability to perform ChIP-seq from 10,000-20,000 (Adli et al). However, Adli et al. has limited application because it requires tens of thousands cells and introduces bias by excessive DNA amplification. The second method aims to reduce the bias in DNA amplification by using the T7 RNA polymerase-based linear DNA amplification, termed LinDA (Shankarananarayanan 2011 and 2012). Although the LinDA method reports the global mapping of sites from as little as 5,000 cells, the results are inconsistent. Furthermore, the reported lower limit of 5,000 cells is still too large of a number that for ChIP-seq in the range of one to a few thousand cells, for example from 20 to 100 cells.

One of the major problems that prevents the use of ChIP-seq when there is a limited number of cells (e.g., one to a few thousand cells) is DNA loss during DNA shearing and subsequently ChIP steps. If the DNA is permanently lost at any step, even the best unbiased DNA amplification will not be useful. Therefore, there is a need to develop a set of techniques that enable efficient DNA recovery from ChIP to allow efficient genome sequencing from a small number of cells.

### Chromatin Immunoprecipitation (ChIP)

The principle underpinning of ChIP is that fragments of the DNA-protein complex that package the DNA in living cells (i.e. the chromatin), can be prepared to retain the specific DNA-protein interactions that characterize each living cell. These chromatin (i.e., the protein-DNA complex) fragments can then be immunoprecipitated using an antibody against the protein in question. The isolated chromatin fraction can then be treated to separate the DNA and protein components, and the identity of the DNA fragments isolated in connection with a particular protein (ie. the protein against which the antibody used for immunoprecipitation was directed), can then be determined by Polymerase Chain Reaction (PCR) or other technologies used for identification of DNA fragments of defined sequence.

ChIP generally involves the following three key steps:—(i) isolation of chromatin to be analyzed from cells; (ii) immunoprecipitation of chromatin using an antibody; and (iii) DNA analysis. While the skilled artisan will appreciate that there are various methods for performing ChIP, the following example is a general overview of the standard principles behind ChIP.

ChIP generally comprises a step of isolating chromatin from the biological sample of cells. Once the cells are harvested, their nuclei are extracted. Following release of the nuclei, the nuclei are digested in order to release the chromatin. In embodiments, where the method comprises use of NChIP (described below), the chromatin is isolated using nuclease digestion of cell nuclei by standard procedures. For example, micrococcal nuclease can be added in the digestion. In embodiments, where the method comprises use of XChIP (described below), the chromatin is crosslinked. For example, the chromatin may be crosslinked by addition of a suitable cross-linking agent, such as formaldehyde. Thereafter, the chromatin is fragmented. Fragmentation may be carried out by sonication. However, formaldehyde may be added after fragmentation, and then followed by nuclease digestion. Alternatively, UV irradiation may be employed as an alternative crosslinking technique.

After fragmentation and crosslinking, the proteins are immobilized on the chromatin and the protein-DNA complex can be immunoprecipitated. Hence, once the chromatin has been isolated, the method comprises a step of immunoprecipitating the chromatin. Suitable techniques for the immunoprecipitation step will also be known to skilled technician, and the Examples describe a method for how this may be achieved. Immunoprecipitation can be carried out upon addition of a suitable antibody against the protein in question. It will be appreciated that the suitable antibody will depend on what type of epigenetic analysis is being carried out (i.e. the gene expression that is being analyzed).

Epigenetic analysis is the study of various changes (known as epigenetic marks) to the DNA of a cell, which tend to result in expression or silencing of genes. It should be appreciated that the method according to the invention may be used to assay epigenetic modifications of any sort, on any gene, or region of the genome of any cell type of interest. Examples of epigenetic marks, which may be caused by modification of DNA in the sample include histone protein modification, non-histone protein modification, and DNA methylation.

Accordingly, for example, the antibody used in the immunoprecipitation step may be immunospecific for non-histone proteins such as transcription factors, or other DNA-binding proteins. Alternatively, for example, the antibody may be immunospecific for any of the histones H1, H2A, H2B, H3 and H4 and their various post-translationally modified isoforms and variants (eg. H2AZ). Alternatively, for example, the antibody may be immunospecific for enzymes involved in modification of chromatin, such as histone acetylases or deacetylases, or DNA methyltransferases. Furthermore, histones may be post-translationally modified in vivo, by defined enzymes, for example, by acetylation, methylation, phosphorylation, ADP-ribosylation, sumoylation and ubiquitination. Accordingly, the antibody may be immunospecific for any of these post-translational modifications.

Following the immunoprecipitation step, the method generally comprises a step of purifying DNA from the isolated protein/DNA fraction. This may be achieved, for example, by the standard technique of phenol-chloroform extraction or by any other purification method known to one of skill in the art.

Following the purification step, the DNA fragments isolated in connection with the protein is analyzed by PCR. For example, the analysis step may comprise use of suitable primers, which during PCR, will result in the amplification of a length of nucleic acid. The skilled artisan will appreciate that the method according to the invention may be applied to analyze epigenetic modifications on any gene or any region of the genome for which specific PCR primers are prepared.

The ChIP technique has two major variants that differ primarily in how the starting (input) chromatin is prepared. The first variant (designated NChIP) uses native chromatin prepared by micrococcal nuclease digestion of cell nuclei by standard procedures. However, NChIP is not useful for analyzing non-histone proteins because selective nuclease digestion may bias input chromatin and nucleosomes may rearrange during digestion.

The second variant (designated XChIP) uses chromatin cross-linked by addition of formaldehyde to growing cells, prior to fragmentation of chromatin (e.g., fragmentation by sonication). As an alternative to formaldehyde, UV irradiation has been successfully employed as an alternative cross-linking technique. However, XChIP is often extremely inefficient can produce false results. For example, XChIP cross-linking may fix (and thereby amplify) transient interactions between proteins and genomic DNA. Furthermore, antibody specificity may be compromised by chemical changes in the protein that it recognises, induced by the cross-linking procedure, in XChIP.

Furthermore, a major problem with NChIP and XChIP is that they both require at least 10⁶ cells to be able to generate sufficient quantities of chromatin for the technique to work (Nature Genetics, 2005, 37, 1194-1200). Such a high number of cells is achievable with cultured cells, but is impossible with material from sources of low numbers of cells, for example, the early embryo, with a typical ICM comprising less than 60 cells (human) or 20 cells (mouse). For this key reason, ChIP and ChIP-seq are limited to samples of large cell populations, thereby preventing widespread epigenetic analysis of primary cells that have not been cultured or immortalized. Accordingly, because epigenetic changes occur in response to environmental cues, it is not possible to study the epigenetic mechanisms that drive differentiation and cellular changes in vivo using cultured cells (in vitro). In other words, the only way of truly understanding the epigenetic state of cells when in their natural state in an organism, is to study the cells that have been directly extracted (biopsied) from the organism and not expose the cells to artificial conditions in in vitro culture (i.e., propagating the small number of primary cells to at least 10⁶ cells in in vitro culture) which may cause epigenetic modifications.

There are three primary sources of DNA loss during ChIP: sonication, immunoprecipitation, and elution of ChIP DNA from beads. To protect the DNA of interest from loss, it is important to add carrier DNA that can be processed together with the DNA of interest through successive steps of ChIP.

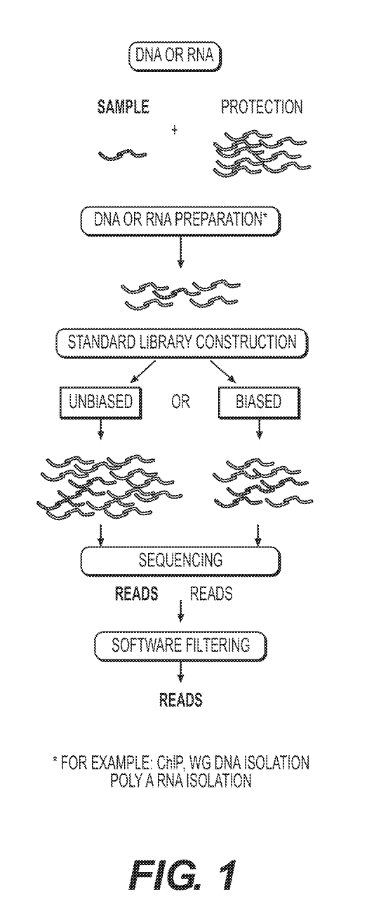

In certain embodiments, the invention described herein encompasses a method of adding biotinylated carrier DNA that is processed with the DNA of interest during ChIP to prevent loss of DNA of interest. As used herein, the method of preventing loss and increasing recovery of the DNA of interest is referred to as “Recovery via Protection” or “RePro” or “RePro ChIP-Seq.” A diagram of RePro is provided in FIG. 1.

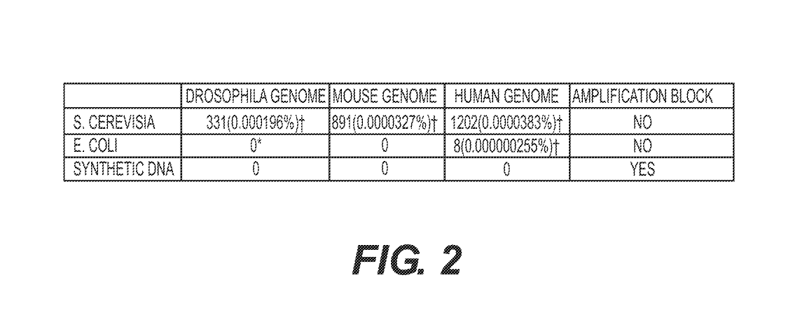

Repro can be performed by mixing a large number of crossed linked cells from a divergent species with the small number of cells of interest. In certain embodiments, the cells from a divergent species are mammalian cells (e.g., human cells, mouse cells, rat cells, hamster cells, feline cells, canine cells, and primate cells), insect cells (e.g., Drosophila cells), bacterial cells (e.g., E. coli cells), or yeast cells (e.g., S. cerevisiae) (FIG. 2).

In specific embodiments, E. coli cells can be used as the cells from a divergent species in RePro of Drosophila, mouse, or human cells. In specific embodiments, S. cerevisiae cells can be used as the cells from a divergent species in RePro of Drosophila, mouse, or human cells.

In one specific embodiment, yeast cells are used for epigenetic profiling of histone H3 lysine 4 or lysine 9 methylations (H3K4me or H3K9me, respectively) because the same antibodies can be used to ChIP the chromatin that exhibit these epigenetically modified histone marks in yeast, Drosophila, mouse, and humans.

### Analyte Biological Sample

In certain embodiments, the methods described herein comprise carrying out ChIP-seq using less than one million cells, less than 900,000 cells, less than 800,000 cells, less than 700,000 cells, less than 600,000 cells, less than 500,000 cells, less than 400,000 cells, less than 300,000 cells, less than 200,000 cells, less than 90,000 cells, less than 80,000 cells, less than 70,000 cells, less than 60,000 cells, less than 50,000 cells, less than 40,000 cells, less than 30,000 cells, less than 20,000 cells, or less than 10,000 cells as the analyte biological sample.

In certain embodiments, the methods described herein comprise carrying out ChIP-seq using approximately 20,000 cells, approximately 19,000 cells, approximately 18,000 cells, approximately 17,000 cells, approximately 16,000 cells, approximately 15,000 cells, approximately 14,000 cells, approximately 13,000 cells, approximately 12,000 cells, approximately 11,000 cells, approximately 10,000 cells, approximately 9,500 cells, approximately 9,000 cells, approximately 8,500 cells, approximately 7,500 cells, approximately 7,000 cells, approximately 6,500 cells, approximately 6,000 cells, approximately 5,500 cells, approximately 5,000 cells, approximately 4,500 cells, approximately 4,000 cells, approximately 3,500 cells, approximately 3,000 cells, approximately 2,500 cells, approximately 2,000 cells, approximately 1,900 cells, approximately 1,800 cells, approximately 1,700 cells, approximately 1,600 cells, approximately 1,500 cells, approximately 1,400 cells, approximately 1,300 cells, approximately 1,200 cells, approximately 1,100 cells, approximately 1,000 cells, approximately 950 cells, approximately 900 cells, approximately 850 cells, approximately 800 cells, approximately 750 cells, approximately 700 cells, approximately 650 cells, approximately 600 cells, approximately 550 cells, approximately 500 cells, approximately 450 cells, approximately 400 cells, approximately 350 cells, approximately 300 cells, approximately 250 cells, approximately 200 cells, approximately 150 cells, approximately 100 cells, approximately 90 cells, approximately 80 cells, approximately 70 cells, approximately 60 cells, approximately 50 cells, approximately 40 cells, approximately 35 cells, approximately 30 cells, approximately 25 cells, approximately 20 cells, approximately 15 cells, approximately 10 cells, 9 cells, 8 cells, 7 cells, 6 cells, 5 cells, 4 cells, 3 cells, 2 cells, or 1 cell as the analyte biological sample.

In certain embodiments of the invention, the method comprises carrying out ChIP on less than 5,000 cells, less than 1,000 cells, less than 500 cells, less than 100 cells, less than 75 cells, less than 50 cells, or less than 25 cells as the analyte biological sample.

Furthermore, it is estimated that one cell contains about 6×10³ ng DNA per cell and equal amounts of DNA and protein in chromatin. Therefore, the method according to the invention comprises carrying out ChIP on as little as 6×10³ ng DNA, or about 12×10³ ng chromatin (equating to mass of DNA or chromatin in 1 cell).

Accordingly as described above, current use of ChIP in epigenetic analyses requires a minimum of at least a million cells and usually much more, thereby restricting its experimental or diagnostic use to cultured cell models or to situations where only large numbers of cells (i.e. at least a million cells) are available. Hence, the methods described herein provide unexpected results of ChIP-seq using a small number of cells (as few as 20 cells or even as few as 1 cell).

### Recovery via Protection (RePro)

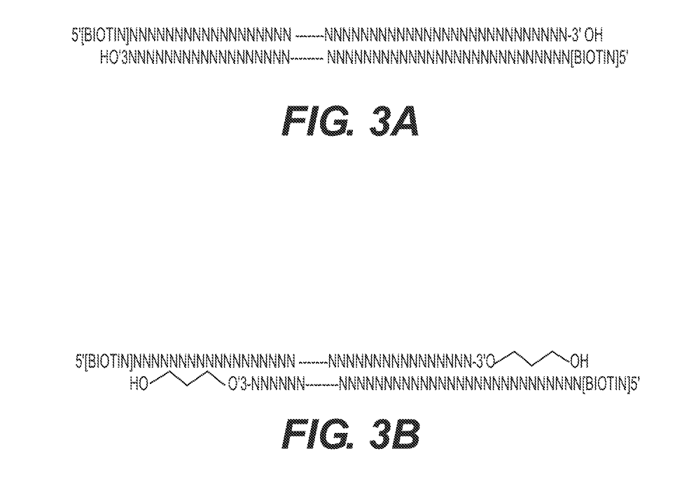

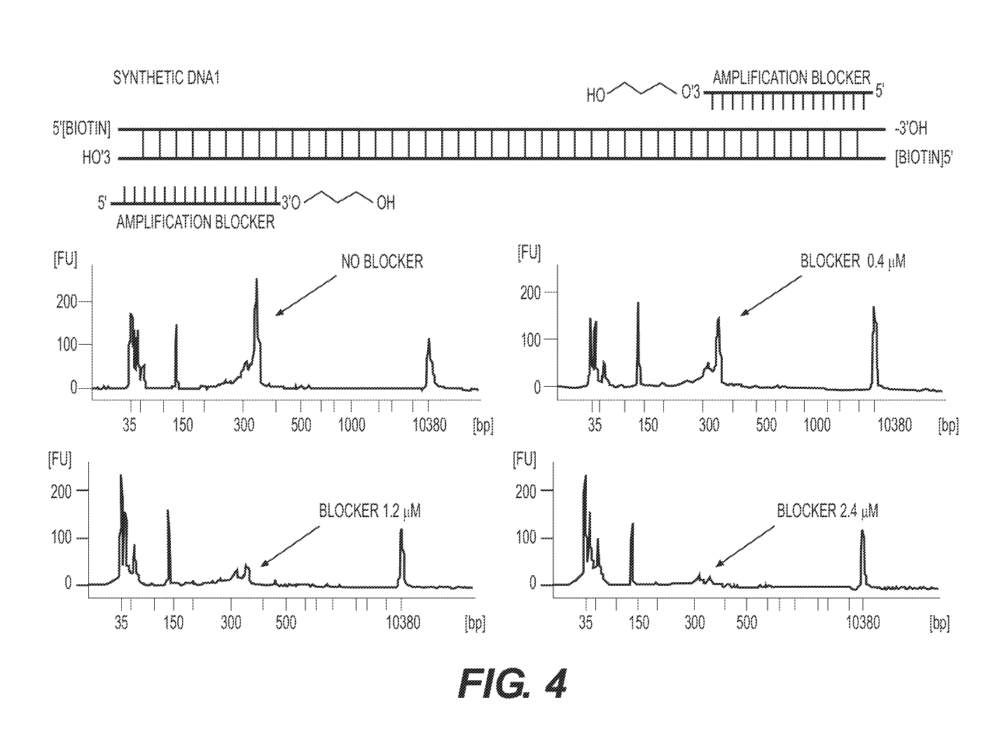

RePro is a ChIP-seq method wherein carrier DNA is added as a bulking agent to decrease DNA loss during ChIP-seq of a small number of cells. The carrier DNA is an oligomer that is approximately 200 base pairs to 300 base pairs in length that are 5′ biotinylated (“DNA1”) (FIG. 3A and FIG. 4). In one embodiment, there is no overlap in the DNA1 sequence and the DNA from the cells of interest.

DNA1 is mixed with the cells of interest for bisulfate conversion or genomic DNA isolation.

For ChIP, after fragmentation of the chromatin, DNA1 is added. Both the chromatin of interest and the DNA1 can then be precipitated using beads that are coupled to agents that recognize specific modifications on chromatin, DNA, or specific proteins bound to the chromatin. For example, the beads can be conjugated to antibodies that specifically bind to the specific modifications on chromatin, DNA, or specific proteins bound to the chromatin.

In one embodiment, streptavidin beads can be used to isolate the biotinylated DNA1.

In another embodiment, in place of the streptavidin beads or in combination with the streptavidin beads, blocking primers are added. The blocking primers consist of DNA sequences that are complementary to the ends of DNA1. The blocking primers, by annealing to the DNA1, prevent PCR amplification of the DNA1.

In another embodiment, DNA1 can be bound to streptavidin that is coupled to unimmunized antibody before adding to the cell. Then, the same protein-A or secondary antibody coupled beads can be used to immunoprecipate both the chromatin of interest and DNA1.

In an alternate embodiment, the DNA1 can be extracted from the mixture prior to PCR.

After the blocking primers are added, the DNA can be amplified using methods of traditional and second generation sequencing known to one of skill in the art.

Because the sequence of DNA1 is known, the remaining DNA1 (and any DNA1 that is amplified as background during the PCR) can be subtracted out post sequencing to provide a clean read of the DNA of interest using software known to one of skill in the art.

### Recovery Via Protection and Favored Amplification (RePam)

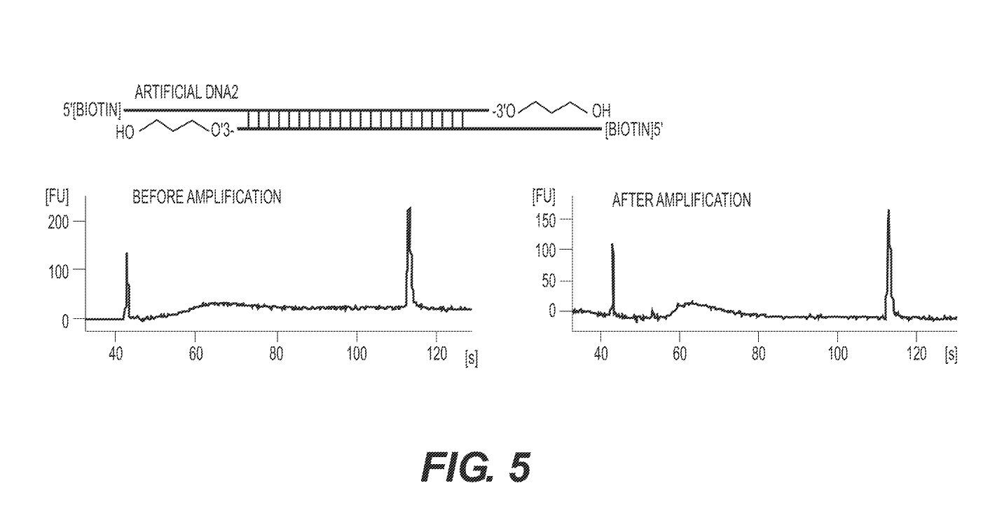

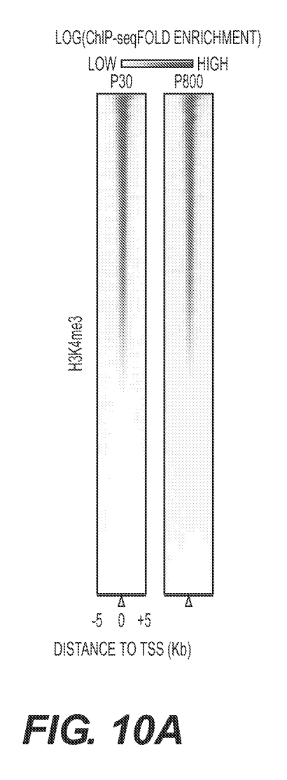

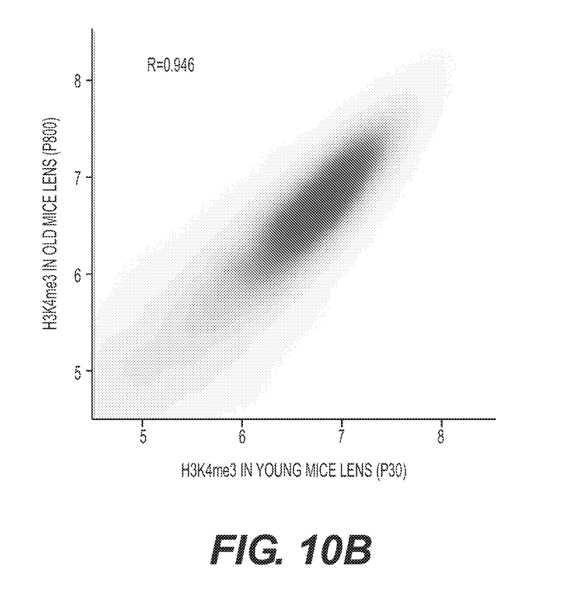

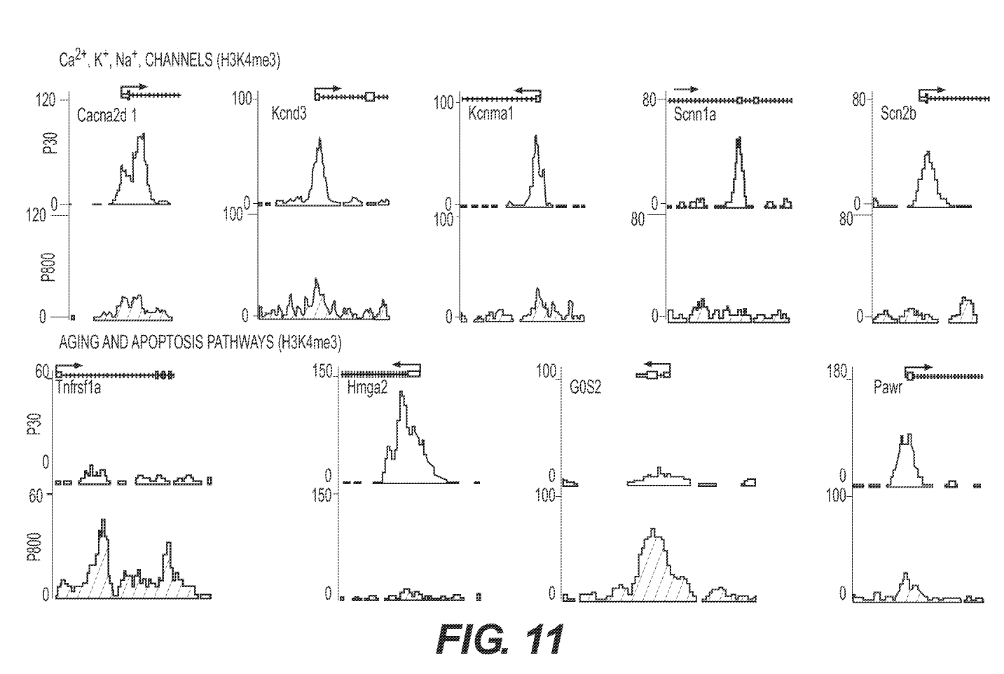

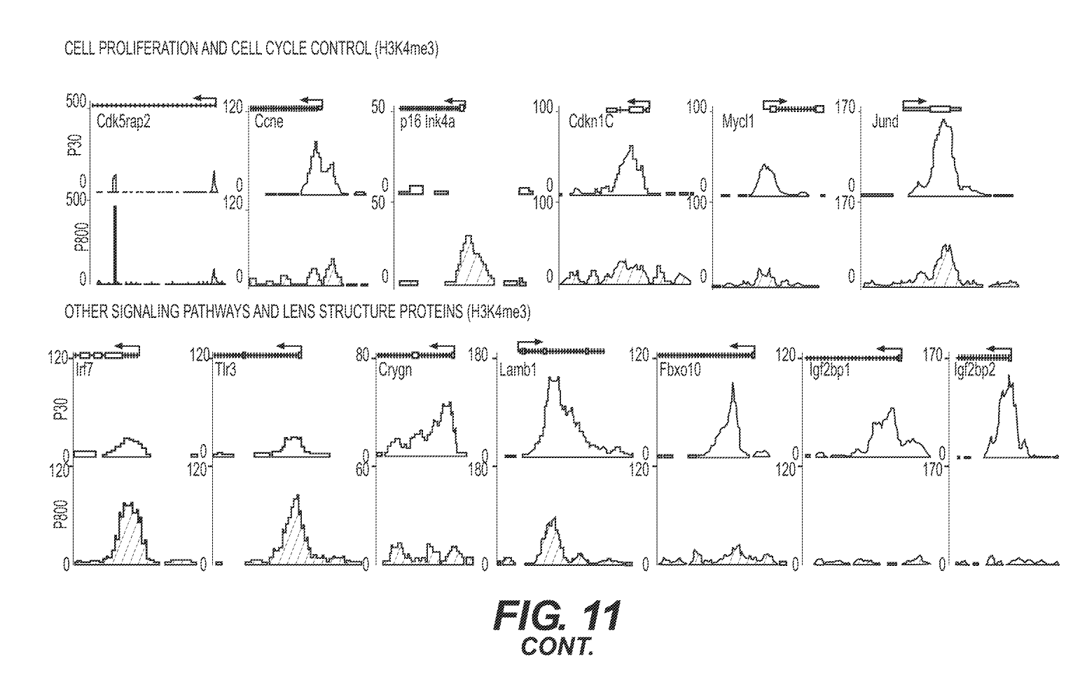

RePam is a ChIP-seq method wherein carrier DNA is added as a bulking agent to decrease DNA loss during ChIP-seq of a small number of cells. The carrier DNA is an oligomer that is approximately 200 base pairs to 300 base pairs in length that are 5′ biotinylated, contain 5′ overhangs, and contain 3′ Spacer 3 modifications on both ends (“DNA2”) (FIG. 3B and FIG. 5 and FIG. 10). The 5′ overhangs and 3′ Spacer 3 modifications prevent amplification of the DNA2 during PCR. In one embodiment, there is no overlap in the DNA2 sequence and the DNA from the cells of interest.

DNA2 is mixed with the cells of interest for bisulfate conversion or genomic DNA isolation.

For ChIP, after fragmentation of the chromatin, DNA2 is added. Both the chromatin of interest and the DNA2 can then be precipitated using beads that are coupled to agents that recognize specific modifications on chromatin, DNA, or specific proteins bound to the chromatin. For example, the beads can be conjugated to antibodies that specifically bind to the specific modifications on chromatin, DNA, or specific proteins bound to the chromatin.

In one embodiment, streptavidin beads can be used to isolate the biotinylated DNA1.

In another embodiment, DNA2 can be bound to streptavidin that is coupled to unimmunized antibody before adding to the cell. Then, the same protein-A or secondary antibody coupled beads can be used to immunoprecipate both the chromatin of interest and DNA2.

For RePam, unlike RePro, blocking primers are not needed because DNA2 is designed to prevent amplification. Accordingly, DNA can be amplified using methods of traditional and second generation sequencing known to one of skill in the art without extracting the DNA2 or blocking the DNA2.

Because the sequence of DNA2 is known, the remaining DNA2 (and any DNA2 that is amplified as background during the PCR) can be subtracted out post sequencing to provide a clean read of the DNA of interest using software known to one of skill in the art.

ChIP-Seq Using Carrier DNA from a Divergent Organism

ChIP-seq can be optimized for a small number of cells by using carrier DNA from a divergent organism. Using this method carrier DNA is added as a bulking agent to decrease DNA loss during ChIP-seq of a small number of cells.

With this method, cells of interest are mixed with cells of a divergent species. In certain embodiments, the cells of a divergent species are yeast or E. coli cells. In certain embodiments, the cells of interest are mouse or human cells. As the cells are sonicated and the DNA is fragmented, the DNA of interest and the DNA of the divergent cells are mixed. Specifically, the DNA of the divergent cells acts as a bulking agent to prevent loss of the DNA of interest and increase yield of the DNA of interest.

As with RePro and RePam, the DNA of interest can be amplified with PCR to assess the epigenetic state of the DNA of interest.

### Accurate Normalization of RNA Reads

As described above for DNA sequencing, there is a similar problem of low RNA yields and the inability to perform massive parallel sequencing of transcripts (RNA-seq). Recent studies (Islam et al. 2011; Hashimshony et al 2012) have shown that it is possible to perform RNA-seq using a single cell. However, the current methods still suffer from the loss of low-abundance transcripts during sample preparation. Such loss of transcripts during the library preparation cannot be remedied by increasing the sequencing depth.

Another serious limitation in the transcriptome analyses by RNA-seq is data normalization. The existing method normalizes each RNA read number against the total or median number of transcript reads, which assumes that the total transcription level to be the same in different samples. However, if the global transcriptional levels are different in different samples, this normalization would produce false identification of transcriptional changes. Alternatively, a known amount of exogenous RNA has been added to RNA-seq samples to allow normalization (Baker, et al.; 2005, Lovén, et al., 2012), but this method requires accurate determination of the number of cells in each sample, which becomes very challenging, if not impossible, when only a few cells are used. Additionally cells at different cell cycle stage have different genomic DNA content that would lead to different transcription levels. Accordingly, this known method is not suitable for comparing transcriptional level between samples with significant cell cycle stage differences. Thus a simpler and more robust method for normalization is needed.

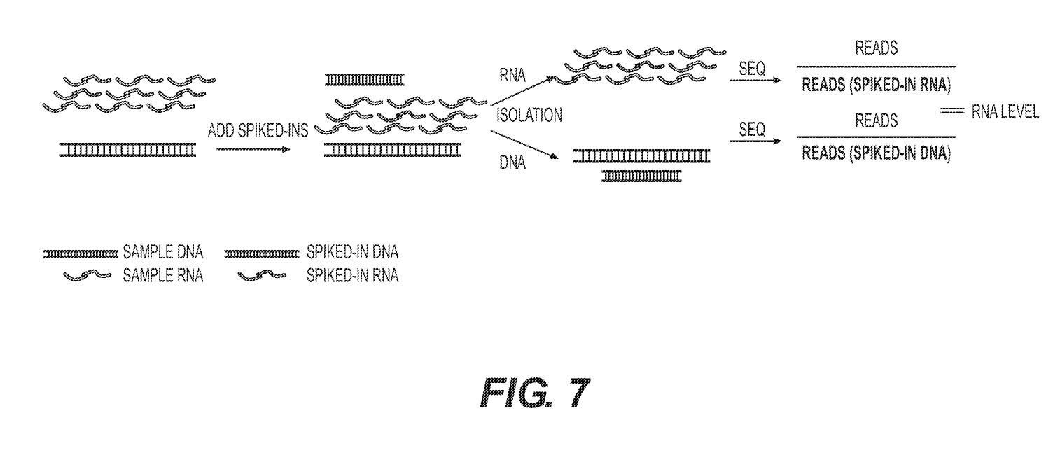

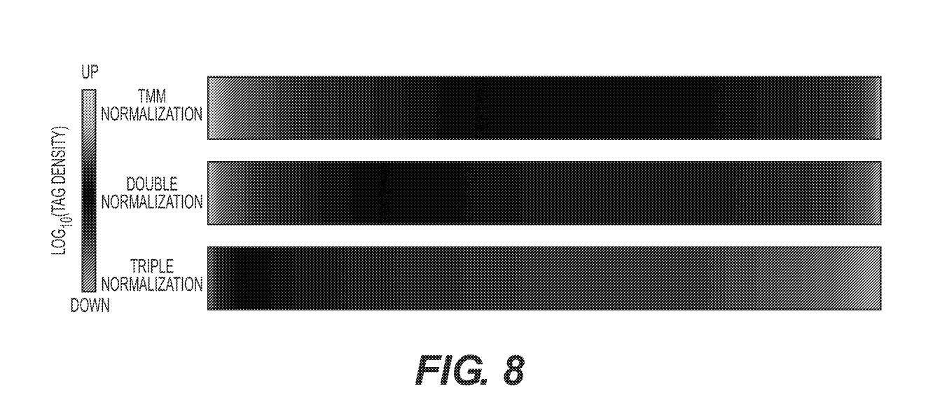

The methods described herein can be used to achieve accurate normalization of RNA reads (FIG. 7 and FIG. 8) and also protect the sample RNA from loss. Specifically, a protection agent which is analogous to the carrier DNA in RePro and RePam, is mixed with a cell(s) of interest. The protection reagent is RNA1. To normalize the sample DNA, a known sequence and quantity of DNA is added to the sample. To normalize the sample RNA, a known sequence and quantity of RNA2 is added to the sample. Both RNA1 and RNA2 are in vitro transcribed RNA with a known but different sequence and with a poly A tail.

DNA and RNA are isolated from the mixture. The DNA mixture containing control DNA and genomic DNA from the cell of interest is subjected to standard genomic DNA library construction and sequencing. To construct sequencing library from the isolated RNA, blocking primers are added to block amplification of the RNA1. The purpose of the blocking primers is to block the amplification of RNA1.

Once the RNA1 is blocked with the blocking primers, amplification can begin. During data processing step, reads from control DNA and control RNA-2 is counted and contaminating reads from the protecting RNA-1 is removed by software. The normalized RNA reads (the ratio of total cellular RNA reads/control RNA-2 reads) is divided by the normalized DNA reads (the ratio of genomic DNA reads/control DNA reads). This number allows the normalization of each transcript reads to genomic DNA level without the need to count the number of cells used in each sample.

### EXAMPLES

### Example 1. Efficiency of DNA Recovery Using RePro

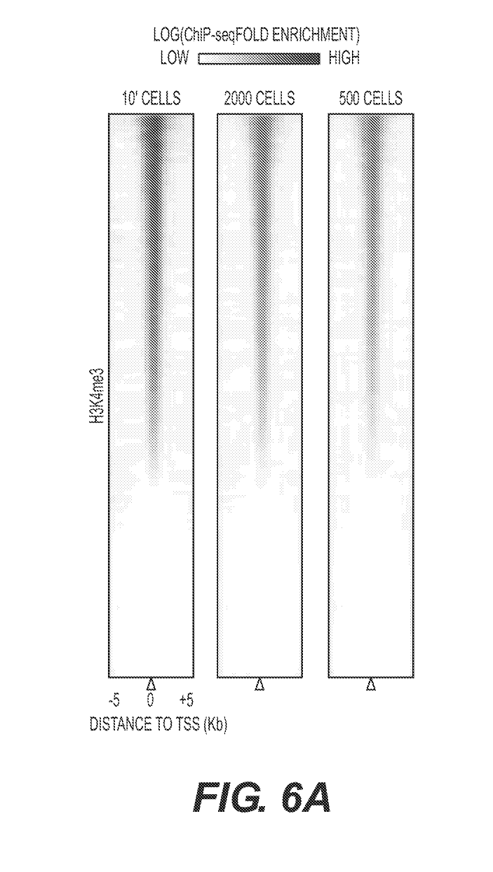

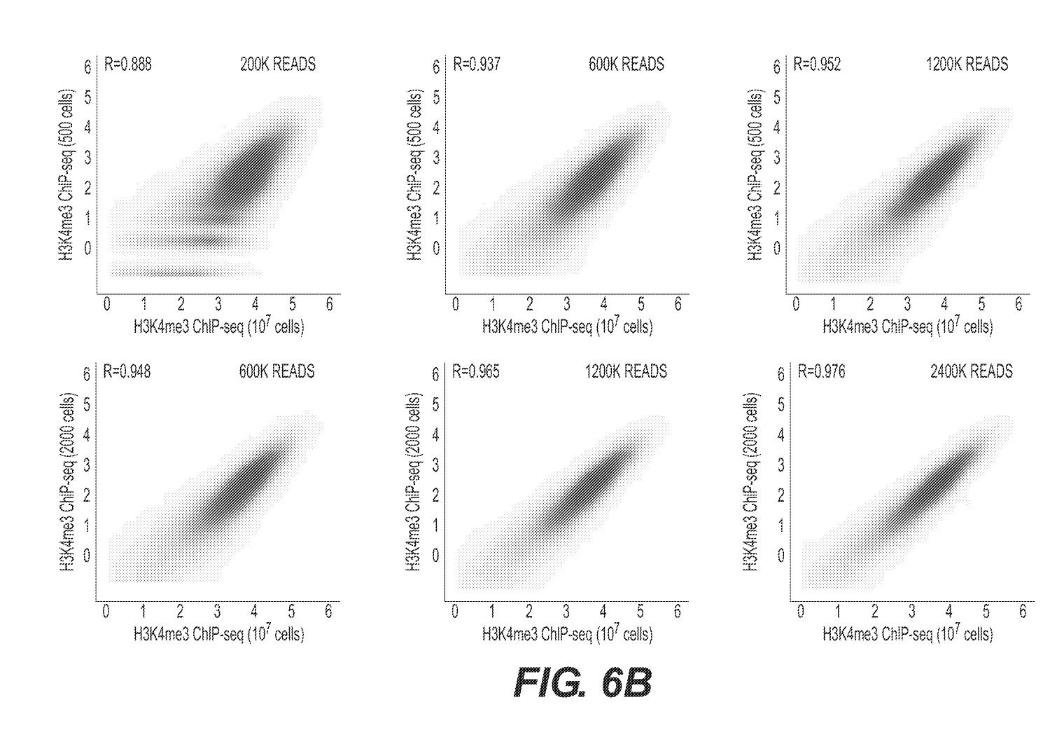

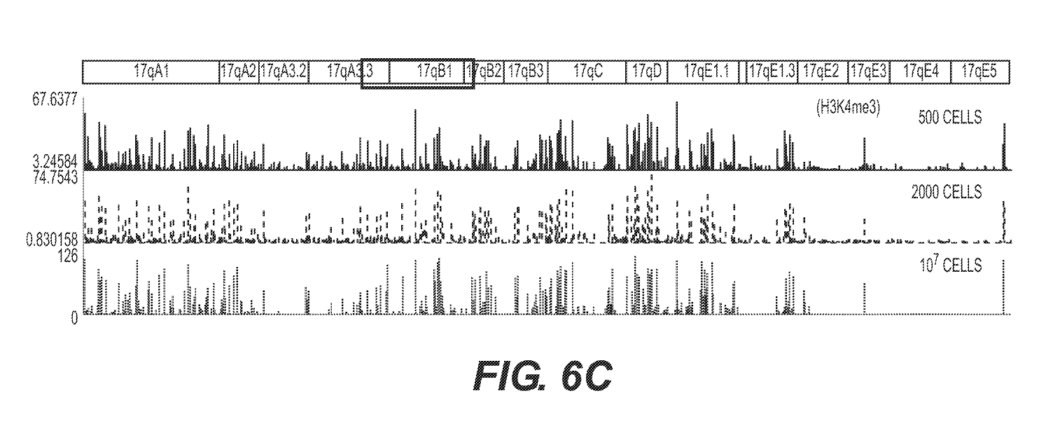

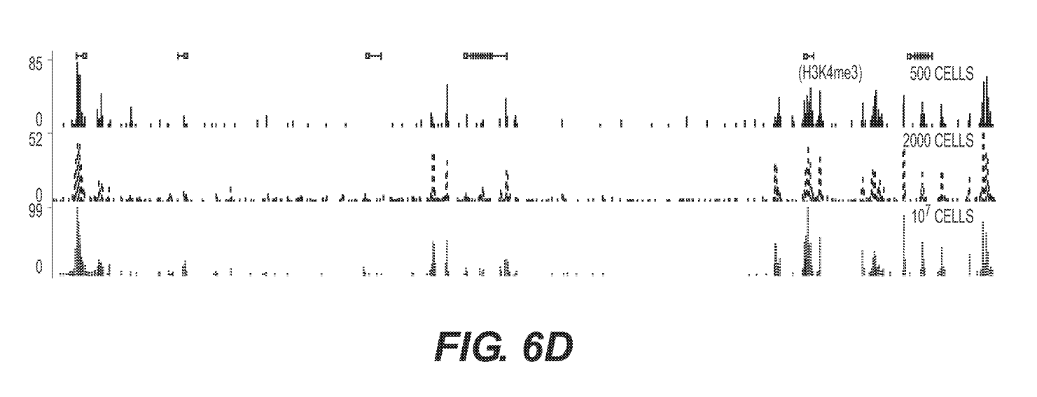

To demonstrate the efficiency of DNA recovery and sequencing quality using RePro, yeast cells were used in RePro ChIP-seq to analyze the H3K4me3 modification in 2000 and 500 mouse embryonic stem cells (ESCs) as compared to standard ChIP-seq of 10 million cells (FIG. 6). Yeast cells were cross linked using formaldehyde and mixed with either 2000 or 500 cross-linked ESCs. Following sonication to break the DNA to 200-300 base pairs, the antibody that recognizes H3K4me3 was used to ChIP the yeast and ESC chromatin carrying the H3K4me3 modifications using the standard ChIP and library building procedures.

By comparing with the standard ChIP-seq of 10 million ESCs, it is shown that RePro ChIP-seq of 500 or 2000 cells uncovered the majority of H3K4me3 modifications in ESCs (correlation coefficiencies, 500 cells: R=0.888; 2000 cells: R=0.948) at the sequencing depth of 200K reads. Importantly, further increasing of read depth up to 1200K led to continuous increasing of H3K4me3 modified DNAs.

Thus, the RePro-ChIP strategy successfully preserved DNA of interest that could be recovered by increasing the depth of sequencing.

### Example 2. Biotinylated DNA Oligos as Carrier DNA

To further broaden the RePro to allow ChIP of any chromatin binding proteins or epigenetic marks, biotinylated DNA oligos were tested (FIG. 4). The streptavidin beads and beads coupled with the specific ChIP antibodies were added to the DNA oligo and chromatin mixture for immunoprecipitation. To block the binding of streptavidin beads to the endogenously biotinylated chromatin proteins, streptavidin was used to block the biotin on these proteins in the cells of interest right after the cells were cross linked using formaldehyde and permeabilized. The excess streptavidin was then blocked. After adding the biotinylated DNA oligos to these cells, they were processed for sonication, immunoprecipitation, and DNA recovery.

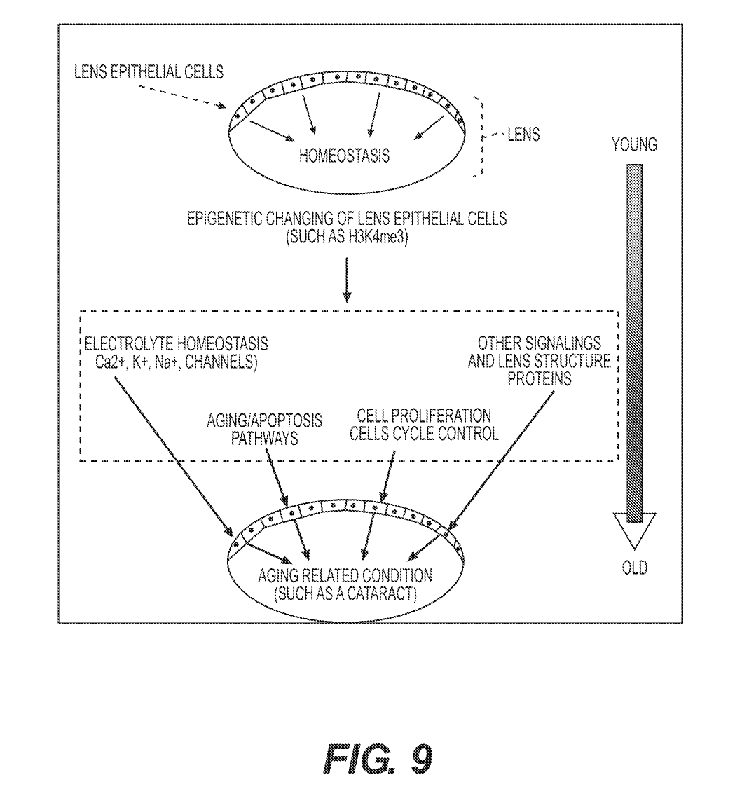

To test the utility of the above methodology, RePro ChIP-seq analyses of H3K4me3 modification was performed in lens epithelial cells from young and old mice (FIG. 8 and FIG. 9). The changes in lens epithelial cells are known to contribute toward cataracts. The ability to map the epigenetic changes associated with aging in these cells should provide insights into the causes of cataract formation. By RePro-ChIP-seq of the lens epithelial cells isolated from one old and one young lens, it was shown that about 200 genes whose H3K4me3 became either up or down-regulated in the old lens epithelial cells compared to the young cells. Importantly, many of these genes are involved in biological processes that have been implicated in the degeneration of lens epithelial cells and cataract formation. These pathways include genes involved in regulating apoptosis, electrolyte homeostasis, and the cell cycle.

Interestingly, two of these genes have already been found in GWAS (genome wide association study) analyses with SNPs associated with predisposition to cataract in human population. It has been suggested that by combining GWAS with EWAS (epigenetic genome wide association study), it may be possible to identify disease-causing/diagnostic genes and gene expression changes with significantly increased accuracy and efficiency. Since accurate EWAS requires a pure cell population that is limited by a very small cell number, it has not been possible to perform EWAS analyses of histone modifications. The above example shows that the methods described herein can open the door to perform EWAS in human disease gene discovery and diagnosis.

### Example 3. Simulation to Determine the Lower Limits of Cell Numbers for Optimum ChIP-seq

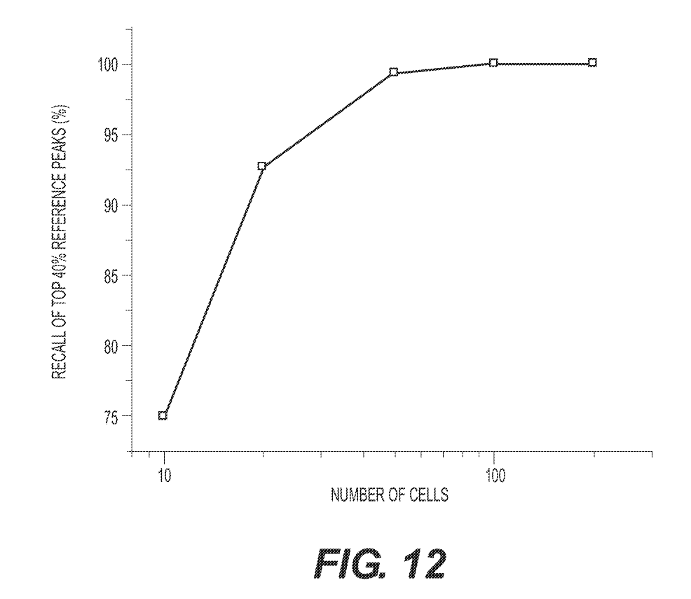

Simulated ChIP-seq reads were performed to determine the lower limit of cell numbers needed to provide optimum sequencing results (FIG. 12).

Simulative ChIP-seq reads were sampled from the genome with binomial distribution according to a 10⁷-cell H3K4me3 ChIP-seq data (Jia 2012). It was assumed that the Oct4 gene H3K4me3 peak, which is among the highest H3K4me3 peaks in the genome, is fully ChIPed, and the probability of generating a read from specific genomic position is in proportion to the ChIPseq tag density at the position and the cell number.

It was assumed that only 10% of input chromatin is recovered, therefore, 10% percent of ChIPed reads were kept in the final library.

Then for each test set of different cell numbers, peaks were called using MACS in variable p value thresholds. The precision and recall were defined as previously described by comparing to another H3K4me3 ChIP-seq data (Mikkelsen 2007). FIG. 12 plots the recall from different number of cells with 80% or higher precision. Based on this simulation, if the chromatin recovery from cells can reach 10% of input, the theoretical limit of the lowest number of cells for RePro and RePam-ChIP-seq is 20.

### Example 4. Comparison of Repro H3K4Me3 Data with Nano-ChIP-Seq and LinDA

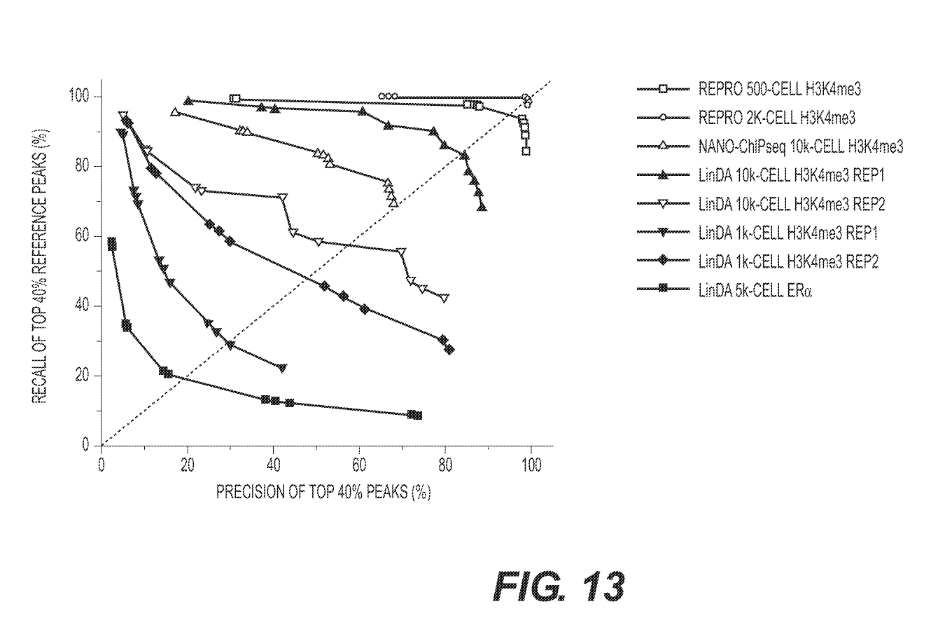

As described herein, there are two existing ChIP-seq methods that claim to be able to perform ChIP-seq from small number of cells. They are called Nano-ChIP-seq and LinDA-ChIP-seq. Analyses of the data from Nano-ChIP-seq and LinDA-seq were performed and the results were compared to the RePro methods described herein (FIG. 13).

The Nano-ChIP-seq method only allows for ChIP-sequencing of 10,000 cells. The data obtained from the LinDA method using 1,000 cells is not very robust and cannot be used for obtaining any useful information. As a result, the LinDA method also uses data obtained from analyzing 10,000 cells.

One criterion for acceptable replicate adopted by the ENCODE project (Landt 2012) is that at least 80% of the top 40% target identified from one replicate should overlap the target of another replicate. This criterion was used to test whether the RePro H3K4me3 data could be accepted as replicate of previous H3K4me3 ChIP-seq data using over 10 million cells (Mikkelsen 2007). “Precision” is defined as the percentage of top 40% peaks identified from the RePro H3k4me3 data that overlaps the previous H3K4me3 peaks, and “recall” as the percentage of top 40% peaks identified from previous H3K4me3 data that overlaps the RePro H3K4me3 peaks. The RePro H3K4me3 ChIP-seq data reached 98.2% precision and 93.7% recall with 500 cells, and almost 100% precision and recall with only 2000 cells (FIG. 13). These results show that the RePro method can reliably recover ChIP-seq peaks with minute amount of starting material. By contrast, the Nano-ChIP-seq data for H3k4me3 with 10,000 cells can only reach 70% and 70% precision and recall level, respectively, which does not meet the 80%/80% criterion. This is probably due to the high bias in the data introduced by more than 30 cycles of PCR. Therefore this method is not suitable for ChIP-sequencing from 10,000 cells.

Similar tests were implemented for LinDA-ChIP-seq by comparing to the reference dataset used in their study. Although LinDA can have precision and recall both over 80% in one experiment using 10,000 cells for H3K4me3 ChIP-seq, another replicate of it gave a much worse result of below 60%-60% precision-recall level, respectively, showing that the method is unstable and not usable, probably due to the complex and time-consuming procedures involving transcription of DNA into RNA and reverse transcription of RNA back into DNA. Moreover, the poor qualities of 1,000 cell H3K4me3 ChIP-seq data and 5,000 cell Era (a transcription factor) data show that LinDA is not capable of generating informative ChIP-seq data from less than 10,000 cells.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present disclosure additionally provides the following methods

1. A method of sequencing genomic DNA from a sample of cells, the method comprising:

a. Fragmenting chromatin in the sample of cells,

b. Adding a carrier DNA to the fragmented chromatin of the sample of cells, wherein the carrier DNA is 5′ biotinylated DNA (“DNA1”),

c. Precipitating the mixture of carrier DNA and fragmented chromatin,

d. Annealing a blocking primer that is complementary to the DNA1,

e. Amplifying the genomic DNA from the sample of cells, and

f. Sequencing the amplified DNA;

wherein the sample of cells comprise between 1 and 20,000 cells, and wherein the blocking primers prevent amplification of the DNA1.

2. The method of 1, wherein the sample of cells are mammalian cells.

3. The method of 2, wherein the mammalian cells are human or mouse cells.

4. The method of any of 1 to 3, wherein the sample of cells are primary cells.

5. The method of any of 1 to 4, wherein sequenced DNA is used to determine the epigenetic signature of the sample of cells.

6. The method of any of 1 to 5, wherein the sample of cells comprises 1 cell.

7. The method of any of 1 to 5, wherein the sample of cells comprises about 20 cells.

8. The method of any of 1 to 5, wherein the sample of cells comprises about 50 cells.

9. The method of any of 1 to 5, wherein the sample of cells comprises about 100 cells.

10. The method of any of 1 to 5, wherein the sample of cells comprises about 1000 cells.

11. The method of any of 1 to 10, wherein the sample of cells is a sample of cancer cells.

12. The method of any of 1 to 10, wherein the sample of cells is a sample of lens epithelial cells.

13. The method of any of 1 to 12, wherein the DNA1 is between 200 base pairs and 300 base pairs in length.

14. The method of any of 1 to 13, wherein the DNA1 is not complementary to the DNA from the sample of cells.

15. The method of any of 1 to 14, wherein the mixture of DNA1 and fragmented chromatin is precipitated with beads.

16. The method of 15, wherein the beads are conjugated to an antibody.

17. The method of 16, wherein the antibody is directed to modifications of the chromatin or to proteins bound to the chromatin.

18. The method of 15, wherein the beads are conjugated to an agent that specifically binds the DNA from the sample of cells.

19. The method of 18, wherein the agent is a DNA strand that is complementary to a portion of the DNA from the sample of cells.

20. A method of sequencing genomic DNA from a sample of cells, the method comprising:

a. Fragmenting the chromatin of the sample of cells,

b. Adding a carrier DNA to the fragmented chromatin of the sample of cells, wherein the carrier DNA is 5′ biotinylated with a 5′ overhang and a 3′ Spacer 3 modification (“DNA2”),

c. Precipitating the mixture of carrier DNA and fragmented chromatin,

d. Amplifying the genomic DNA from the sample of cells, and

e. Sequencing the amplified DNA;

wherein the sample of cells comprise between 1 and 20,000 cells.

21. The method of 20, wherein the sample of cells are mammalian cells.

22. The method of 21, wherein the mammalian cells are human or mouse cells.

23. The method of any of 20 to 22, wherein the sample of cells are primary cells.

24. The method of any of 20 to 23, wherein sequenced DNA is used to determine the epigenetic signature of the sample of cells.

25. The method of any of 20 to 24, wherein the sample of cells comprise 1 cell.

26. The method of any of 20 to 24, wherein the sample of cells comprise 20 cells.

27. The method of any of 20 to 24, wherein the sample of cells comprise 50 cells.

28. The method of any of 20 to 24, wherein the sample of cells comprise 100 cells.

29. The method of any of 20 to 24, wherein the sample of cells comprise 1000 cells.

30. The method of any of 20 to 29, wherein the sample of cells is a sample of cancer cells.

31. The method of any of 20 to 29, wherein the sample of cells is a sample of lens epithelial cells.

32. The method of any of 20 to 31, wherein the DNA2 is between 200 base pairs and 300 base pairs in length.

33. The method of any of 20 to 32, wherein the DNA2 is not complementary to the DNA from the sample of cells.

34. The method of any of 20 to 33, wherein the mixture of DNA2 and fragmented chromatin is precipitated with beads.

35. The method of 34, wherein the beads are conjugated to an antibody.

36. The method of 25, wherein the antibody is directed to modifications of the chromatin or to proteins bound to the chromatin.

37. The method of 34, wherein the beads are conjugated to an agent that specifically binds the DNA from the sample of cells.

38. The method of 37, wherein the agent is a DNA strand that is complementary to the DNA from the sample of cells.

39. A method of sequencing genomic DNA from a sample of cells, the method comprising:

a. Combining a sample of cells of interest with a sample of bulking cells,

b. Fragmenting the chromatin of the cells of interest and the bulking cells,

c. Precipitating the fragmented chromatin of the cells of interest,

d. Amplifying the genomic DNA from the sample of cells, and

e. Sequencing the amplified DNA;

wherein the sample of cells comprise between 1 and 20,000 cells, and wherein the bulking cells are yeast cells or E. coli cells.

40. The method of 39, wherein the sample of cells are mammalian cells.

41. The method of 40, wherein the mammalian cells are human or mouse cells.

42. The method of any of 39 to 41, wherein the sample of cells are primary cells.

43. The method of any of 39 to 42, wherein sequenced DNA is used to determine the epigenetic signature of the sample of cells.

44. The method of any of 39 to 43, wherein the sample of cells comprise 1 cell.

45. The method of any of 39 to 43, wherein the sample of cells comprise 20 cells.

46. The method of any of 39 to 43, wherein the sample of cells comprise 50 cells.

47. The method of any of 39 to 43, wherein the sample of cells comprise 100 cells.

48. The method of any of 39 to 43, wherein the sample of cells comprise 1000 cells.

49. The method of any of 39 to 48, wherein the sample of cells is a sample of cancer cells.

50. The method of any of 39 to 48, wherein the sample of cells is a sample of lens cells.

51. The method of any of 39 to 50, wherein the bulking cells are S. cerevisia.

52. The method of any of 39 to 50, wherein the bulking cells are E. coli.

53. The method of any of 39 to 52, wherein the fragmented chromatin is precipitated with beads.

54. The method of 53, wherein the beads are conjugated to an antibody.

55. The method of 54, wherein the antibody is directed to modifications of the chromatin of the cells of interest or to proteins bound to the chromatin of the cells of interest.

56. The method of 53, wherein the beads are conjugated to an agent that specifically binds the DNA from the cells of interest.

57. The method of 56, wherein the agent is a DNA strand that is complementary to the DNA from the cells of interest.

1. A method of sequencing genomic DNA from a sample of cells, the method comprising:
a. fragmenting chromatin in the sample of cells,
b. adding a carrier DNA to the fragmented chromatin of the sample of cells, wherein the carrier DNA is 5′ biotinylated DNA (“DNAI”),
c. precipitating the mixture of carrier DNA and fragmented chromatin,
d. annealing a blocking primer that is complementary to the DNAI, said blocking primer comprising at least one modification or component that prevents degradation,
e. amplifying the genomic DNA from the sample of cells, and
f. sequencing the amplified DNA;
wherein the sample of cells comprise between 1 and 20,000 cells, and wherein the blocking primers prevent amplification of the DNAI.

2. The method of claim 1, wherein the sample of cells are mammalian cells.

3. The method of claim 2, wherein the mammalian cells are human or mouse cells.

4. The method of claim 1, wherein the sample of cells are primary cells.

5. The method of claim 1, wherein sequenced DNA is used to determine the epigenetic signature of the sample of cells.

6. The method of claim 1, wherein the sample of cells comprises 1 cell, about 20 cells, about 50 cells, about 100 cells, or about 1000 cells.

7-10. (canceled)

11. The method of claim 1, wherein the sample of cells is a sample of cancer cells.

12. The method of claim 1, wherein the sample of cells is a sample of lens epithelial cells.

13. The method of claim 1, wherein the DNAI is between 200 base pairs and 300 base pairs in length.

14. The method of claim 1, wherein the DNAI is not complementary to the DNA from the sample of cells.

15. The method of claim 1, wherein the mixture of DNAI and fragmented chromatin is precipitated with beads.

16. The method of claim 15, wherein the beads are conjugated to an antibody.

17. The method of claim 16, wherein the antibody is directed to modifications of the chromatin or to proteins bound to the chromatin.

18. The method of claim 15, wherein the beads are conjugated to an agent that specifically binds the DNA from the sample of cells.

19. The method of claim 18, wherein the agent is a DNA strand that is complementary to a portion of the DNA from the sample of cells.

20. A method of sequencing genomic DNA from a sample of cells, the method comprising:
a. fragmenting the chromatin of the sample of cells,
b. adding a carrier DNA to the fragmented chromatin of the sample of cells, wherein the carrier DNA is 5′ biotinylated with a spacer containing a modification or component that blocks amplification of the carrier (“DNA2”),
c. precipitating the mixture of carrier DNA and fragmented chromatin,
d. amplifying the genomic DNA from the sample of cells, and
e. sequencing the amplified DNA;
wherein the sample of cells comprise between 1 and 20,000 cells.

21. The method of claim 20, wherein the sample of cells are mammalian cells.

22. The method of claim 21, wherein the mammalian cells are human or mouse cells.

23. The method of claim 20, wherein the sample of cells are primary cells.

24. The method of claim 20, wherein sequenced DNA is used to determine the epigenetic signature of the sample of cells.

25. The method of claim 20, wherein the sample of cells comprise 1 cell, about 20 cells, about 50 cells, about 100 cells, or about 1000 cells.

26-29. (canceled)

30. The method of claim 20, wherein the sample of cells is a sample of cancer cells.

31. The method of claim 20, wherein the sample of cells is a sample of lens epithelial cells.

32. The method of claim 20, wherein the DNA2 is between 200 base pairs and 300 base pairs in length.

33. The method of claim 20, wherein the DNA2 is not complementary to the DNA from the sample of cells.

34. The method of claim 20, wherein the mixture of DNA2 and fragmented chromatin is precipitated with beads.

35. The method of claim 34, wherein the beads are conjugated to an antibody.

36. The method of claim 25, wherein the antibody is directed to modifications of the chromatin or to proteins bound to the chromatin.

37. The method of claim 34, wherein the beads are conjugated to an agent that specifically binds the DNA from the sample of cells.

38. The method of claim 37, wherein the agent is a DNA strand that is complementary to the DNA from the sample of cells.
